(19) United States
(12) Patent Application Publication
(10) Pub. No.: US 2006/0194983 A1
(43) Pub. Date: Aug. 31, 2006

(54) Method for the manufacture of l-threonine-0-(1,1-dimethylethyl)-1,1-dimethylethyl ester

(76) Inventors: Mohammed Siddiqui Jaweed Mukarram, Aurangabad (IN); Krishnajl Upadhye Bhargav, Aurangabad (IN); Deep Virupaksha Hedge, Aurangabad (IN)

Correspondence Address: BIO INTELLECTUAL PROPERTY SERVICES (BIO IPS) LLC, 8509 KERNON CT., LORTON, VA, 22079, US

(21) Appl. No.: 10/564,363

(22) PCT Filed: Sep. 8, 2003
(86) PCT No.: PCT/IB03/03784

§ 371 Date: Feb. 23, 2006

(51) Int. Cl. C07C 227/14

(52) U.S. Cl. 560/170

(57) ABSTRACT

The present invention relates to a new and efficient process for the manufacture of L-Threonine-O-(1,1-dimethylethyl) ester; [H-Thr-(tBu)-O-tBu] of Formula (I). The compound of Formula (I) is a key product for the manufacture of human insulin from porcine or bovine insulin.

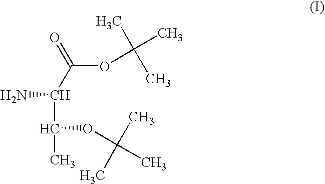

### FIELD OF THE INVENTION

The present invention relates to a new and efficient process for the manufacture of L-Threonine-O-(1,1-dimethylethyl)-1,1-dimethylethyl ester; [H-Thr-(tBu)-O-tBu] of formula I.

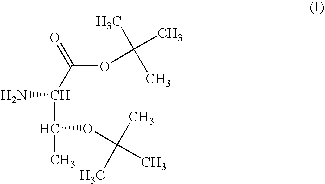

The compound of formula (I) is a key product for the manufacture of human insulin from porcine or bovine insulin.

### BACKGROUND OF THE INVENTION

In the treatment of diabetes mellitus insulin preparations derived from porcine or bovine insulin have generally been used. Human, porcine and bovine insulins exhibit minor differences in their amino acid sequences. Human insulin and pig insulin differ due to the carboxyl terminal amino acid in B30-position of the insulin B chain. In case of human insulin a threonine follows the lysyl radical in B29 whereas in the case of pig insulin alanine comes thereafter. For the production of human insulin the necessary amount of human pancreas gland are not available. Synthetic human insulin has been prepared on a small scale, but it is too expensive, vide Helv. Chim. Acta 57, 2717 and 60, 27.

In addition to total synthesis of human insulin, various semisynthetic processes allow the replacement of alanine by threonine in pig insulin as starting material. U.S. Pat. No. 3,903,068 discloses a desoctapeptide-B23-30 pig insulin obtained by tryptic digestion is linked according to peptide-chemical methods to a protected, synthetic octapeptide of the human insulin sequence B23-30. After linking protective groups are split off and poor yield of human insulin is obtained after tedious chromatographic purification.

Proc. 2^{nd}. Intern. Insulin Symposium 1979, pp. 118-123 or K. Morihara et al., Nature 280, 412-13 (1979) and EP-A No. 0017938 starts from Des-Ala-B30 insulin (pig) and in two step process link with threonine-methyl ester or threonine-tert.-butyl ester by means of trypsin to form the corresponding human insulin ester. After the ester group is deprotected by treatment with sodium hydroxide solution or trifluoroacetic acid, human insulin is obtained in good yield. In addition to the considerably simplified reaction operations, the advantage of the process resides in obtaining human insulin which is free of many impurities and is thus suitable for administration even in immunological problem cases.

U.S. Pat. No. 4,489,159 describes a process for converting human des-B30-insulin into human insulin, Thr-30 ester through amidation with an L-threonine ester in a mixture of water and water miscible solvent in the presence of trypsin and optionally an acid in excess of at least 90% yield.

U.S. Pat. No. 4,601,852 discloses the use of L-Threonine-O-(1,1-dimethylethyl)-1,1-dimethylethyl ester in the manufacture of human insulin from pig insulin by reacting the pig insulin starting material at a pH below its isoelectric point in the presence of trypsin or a trypsin like enzyme in good yield.

Journal of American Chemical Society 85, 201-207 (1963) teaches two different processes for the manufacturing of L-Threonine-O-(1,1-dimethylethyl)-1,1-dimethylethyl ester. In first process N-Carbobenzoxy-L-threonine is coupled with isobutylene to obtain amino protected [H-Thr-(tBu)-O-tBu] in 88% yield. N-Carbobenzoxy-L-Thr-(tBu)-O-tBu is simply treated with 10% palladium on charcoal to get the desired product namely L-Threonine-O-(1,1-dimethylethyl)-1,1-dimethylethyl ester. This process comprises three steps to get H-Thr-(tBu)-O-tBu. Second process comprises the reaction of L-threonine with isobutylene without protecting amino function but the yield obtained is only 35% and also few side products are generated which is a problematic situation in respect to chromatographic purification.

There is need to find an economical, high yield and short step manufacturing process for L-Threonine-O-(1,1-dimethylethyl)-1,1-dimethylethyl ester as the product is very useful for the manufacture of human insulin from pig insulin.

### SUMMARY OF THE INVENTION

According to the present invention a process is provided for the manufacture of L-Threonine-O-(1,1-dimethylethyl)-1,1-dimethylethyl ester [H-Thr-(tBu)-O-tBu]. The manufacturing process for H-Thr-(tBu)-O-tBu comprises the reaction of L-Threonine with Isobutylene in presence of conc. H₂SO₄ in Dimethoxyethane. The reaction mixture is stirred between 0 to 5° C. for 24 hours. The crude reaction mixture is neutralized by a mixture of water and aqueous ammonia followed by extraction with isopropyl ether. Organic layer is concentrated and obtained crude H-Tbr-(tBu)-O-tBu is distilled under reduced pressure to get pure product (99% analyzed by GC).

### DETAILED DESCRIPTION OF THE INVENTION

The present study is directed towards an efficient and single step protection methodology of COOH and OH protected L-Alanine. According to the present invention a process is provided for the manufacture of L-Threonine-O-(1,1-dimethylethyl)-1,1-dimethylethyl ester [H-Thr-(tBu)-O-tBu] of formula (I):

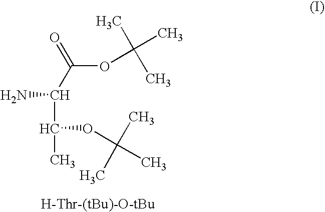

The process employed for the protection of L-Alanine is shown as below:

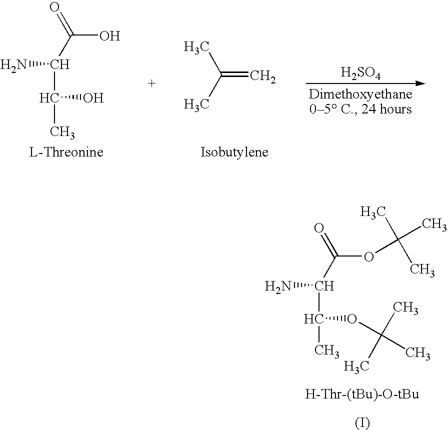

The manufacturing process for H-Thr-(tBu)-O-tBu comprises the reaction of L-Threonine with Isobutylene in presence of mineral acid more preferably conc. Sulfuric acid in Dimethoxyethane. The reaction mixture is stirred for 24 hours between 0 to 5° C. The low temperature is found favorable for lesser side products.

L-Threonine is taken in ethereal solvent specifically in dimethoxyethane and is charged with mineral acid more particularly conc. Sulfuric acid and cooled to −5° C. To the cold solution, Isobutylene is added with maintaining temperature well below 5° C. The molar ratio of Isobutylene with respect to L-Threonine is between about 30 to 50 fold preferably between 35 to 45 fold. The reaction mixture is stirred between 0-5° C. for about 24 hours. After complete protection of —COOH and —OH function of L-Threonine, reaction mixture is poured into a cold solution of water and ammonia (5:1 volume ratio). Neutralized reaction mixture is then extracted with ether more preferably by isopropyl ether. After separation and evaporating the isopropyl ether layer crude H-Thr-(tBu)-O-tBu is obtained in 92% pure form (analyzed by GC). Crude H-Thr-(tBu)-O-tBu is distilled under vacuum (1 mm Hg) and fraction boiling between 75 to 95° C. is collected (4.2 kg), GC purity 99%.

The protecting group employed in the present invention is well known tertiary butyl group. The tertiary butyl group in the present study is very specific in the protection of carboxylic and alcoholic function of L-Alanine leaving amino group free. For selective protection of L-Alanine dimetoxyethane solvent is found most suitable as protected stuff is found in more than 92% pure in crude form. Finally, the desired product is obtained in about 99% pure form after vacuum distillation.

Purified H-Thr-(tBu)-O-tBu is characterized by IR spectroscopy, proton NMR (in CDCl₃) and Mass Spectroscopy. The spectral values are shown in Table 1.

TABLE 1
IR, Proton NMR and Mass Spectral values
of L-Threonine-O-(1,1-dimethylethyl)-1,1-dimethylethyl
ester.

| | |
|---|---|
| IR Peak Values | 3386.8 and 3317.3 cm⁻¹ for —NH₂ Stretching |
| | 1732 cm⁻¹ for —C═O Stretching |
| Proton NMR Peak | 3.85, 3.10, 1.70, 1.45, 1.20 and 1.15. |
| Values | |
| (CDCl₃, δ ppm) | |
| Molecular Mass | 232 [M^{+.} + 1] |
| Peak Value (M/Z) | |

### EXAMPLE 1

### Preparation of L-Threonine-O-(1,1-dimethylethyl)-1,1-diinethylethyl ester; [H-Thr(tBu)-O-tBu], (TBEE)

L-Threonine (5.0 kg, 42.01 Mole) and Dimethoxyethane (100 Ltr) are charged into 250 Ltr capacity glass lined reactor and cooled to −5° C., concentrated sulfuric acid (25 kg, 250.10 Mole) and Isobutylene (94 kg, 1678.57 mole) is then added with maintaining temperature below 5° C. Reaction mixture is stirred between 0 to 5° C. for 24 hours and poured into cold (0° C.) mixture of water (150 Ltr) and ammonia (30 Ltr). It is then extracted using isopropyl ether. Isopropyl ether extract is then concentrated under vacuum to get crude TBEE (4.56 kg) having 92% purity (GC analysis). Crude TBEE is distilled under vacuum (1 mm of Hg) and fraction boiling between 75-95° C. is collected (4.2 kg), GC Purity 99%. IR (Neat Liquid Film) 3386.8 & 1732 cm⁻¹ is for —NH₂ stretching and 1732 cm⁻¹ is for —C═O stretching. Proton Nuclear Magnetic Resonance peaks (δ ppm in CDCl₃) are observed at 3.85 (m, 1H), 3.10 (d, 1H), 1.7 (Br, 2H), 1.45 (s, 9H), 1.2 (d, 3H) and 1.15 (s, 9H). Mass m/z=232 [M⁺+1].

We claim:

1. A process for the preparation of L-Threonine-O-(1,1-dimethylethyl)-1,1-dimethylethyl ester of formula (I) 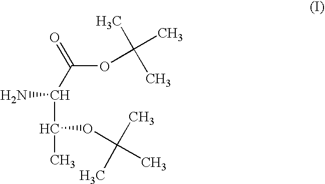
comprising:
A) contacting a compound having the formula 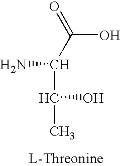 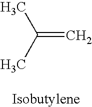
C) in presence of mineral acid in an ether solvent for sufficient time; and
D) purifying the L-Threonine-O-(1,1-dimethylethyl)-1,1-dimethylethyl ester.

2. A process according to claim 1, wherein molar ratio of L-Threonine and Isobutylene is between about 1:30 to 1:50 more preferably between about 1:35 to 1:45.

3. The process of claim 1, wherein the solvent is an ether solvent.

4. The process of claim 3, wherein the solvent is dimethoxyethane.

5. A process according to claim 1, wherein acid is mineral acid.

6. The process of claim 5, wherein mineral acid is sulfuric acid.

7. The process of claim 6, wherein sulfuric acid is concentrated.

8. The process of claim 7, wherein concentrated sulfuric acid is added between about −10 to 15° C.

9. A process according to claim 1, wherein reaction mixture is stirred more than 10 hours.

10. The process of claim 9, wherein the temperature of the reaction mixture is between about −10 to 20° C.

11. A process according to claim 10, wherein the reaction mixture after the formation of product of formula (I) is poured into a mixture of water and ammonia.

12. The process of claim 11, wherein water and ammonia mixture comprises between about 1:1 to 10:1.

13. The process of claim 12, wherein water and ammonia mixture is cold.

14. The process of claim 13, wherein temperature of the water and ammonia mixture is between about 0 to 15° C.

15. A process according to claim 11, wherein an organic solvent is added to the crude product already poured into water and ammonia mixture.

16. The process of claim 15, wherein added organic solvent is an ether.

17. The process of claim 16, wherein ether is Dimethoxyethane.

18. The process of claim 17, wherein after extraction Dimethoxyetane layer is concentrated to get crude compound of Formula (I) in more than 90% pure form.

19. The process of claim 18, wherein crude product is purified by vacuum distillation.

20. The process of claim 19, wherein pressure is reduced to 1 mm of Hg during distillation.

21. The process of claim 20, wherein pure fraction is collected between about 65 to 105° C.

22. The process of claim 21, wherein pure fraction is collected more preferably between about 75 to 95° C.

23. A process according to claim 1, wherein purity of formula (I) compound is about 99%.
